(19) United States
(12) Patent Application Publication
(10) Pub. No.: US 2012/0277569 A1
(43) Pub. Date: Nov. 1, 2012

(54) Optic Characteristic Measuring System and Method

(76) Inventor: Josh N. Hogan, Los Altos, CA (US)

(21) Appl. No.: 13/459,168

(22) Filed: Apr. 28, 2012

Related U.S. Application Data
(60) Provisional application No. 61/518,053, filed on Apr. 30, 2011.

(51) Int. Cl. A61B 3/16

(52) U.S. Cl. 600/400

(57) ABSTRACT

The invention teaches a method, apparatus and system for measuring bio-medical attributes of the eye, such as internal or intraocular pressure. The invention enables taking measurements of the relative location of various surfaces of components of the eye under different conditions. The invention provides for applying a pressure disturbance to the eye acoustically and, using non-invasive optical techniques to perform measurements of vibrations or measurements of the time varying relative location of one or more surfaces or structures in a manner correlated with the pressure disturbance.

### CROSS REFERENCES TO RELATED APPLICATIONS

This application, docket number CI120429US claims priority from US provisional application 61/518053, docket number CI110429PR, of the same title and by the same inventor, the entirety of which is incorporated by reference as if fully set forth herein.

This application relates to U.S. utility application with Ser. No. 12/800,836 filed on 23 May 2010 titled Precision Measuring System, which is a continuation in part of U.S. utility application with Ser. No. 11/048694, filed on Jan. 31, 2005 titled “Frequency Resolved Imaging System”, the contents of both of which are incorporated by reference as if fully set forth herein. This application also relates to U.S. utility application Ser. No. 11/025,698 filed on Dec. 29, 2004 titled “Multiple reference non-invasive analysis system”, the contents of which are incorporated by reference as if fully set forth herein. This application also relates to U.S. utility application Ser. No. 10/949,917 filed on Sep. 25, 2004 titled “Compact non-invasive analysis system”, the contents of which are incorporated by reference as if fully set forth herein.

### GOVERNMENT FUNDING

None

### FIELD OF THE INVENTION

The invention relates to non-invasive optical imaging, measurement and analysis of targets, and, more specifically, targets including biological tissue structures or components of the eye, the living eye in particular. The invention includes monitoring or measuring physical characteristics of the eye under controlled conditions so as to monitor for or measure characteristics such as internal pressure, or aspects related to a malignant condition or the propensity to develop a malignant condition, such as glaucoma.

### BACKGROUND OF THE INVENTION

Non-invasive imaging and analysis is a valuable technique for acquiring information about systems or targets without undesirable side effects, such as damaging the target or system being analyzed. In the case of analyzing living entities, such as human tissue, undesirable side effects of invasive analysis include the risk of infection along with pain and discomfort associated with the invasive process.

In the particular case of non-invasive in-vivo imaging and analysis of biological tissue structures or components, such as structures or components of the eye, it is desirable to measure the physical size of structures or components of the eye under various conditions, for example to measure internal pressure of the eye, or to monitor for the onset of glaucoma or for other ophthalmic related purposes. A non-invasive method with increased precision enables more accurate monitoring of conditions of the eye.

Eye disorders are typically monitored by complex analysis systems related to the medical field of ophthalmology. Such systems include tonometers that are used for measuring intraocular pressure and various types of optical analysis systems that optically measure or monitor physical aspects of components of the eye.

Failure to detect and treat eye disorders at an early stage can result in irreversible damage to the eye leading to impaired vision or complete loss of vision. Such negative impact on vision has significant adverse consequences on quality of life and medical costs.

A method of measuring intraocular pressure non-nvasively is described in U.S. Pat. NO. 5,373,595. The approach uses acoustic techniques to stimulate physical vibrations in the eye and uses a non-invasive optical technique to measure the resulting vibrations in the eye. Such vibrations have resonant frequencies whose magnitude are related to intraocular pressure, thereby enabling a technique for measuring intraocular pressure non-invasively.

Optical coherence tomography low coherence reflectormetry emerged as a technique for measuring properties of the eye. Such techniques are described in patents, such as, U.S. Pat. No. 5,321,501 and papers, such as, “Optical coherence-domain reflectometry: a new optical evaluation technique” by Youngquist et Al. Optics Letters/Vol. 12, No. 3/March 1987 Page 158.

Conventional optical coherence tomography systems related to ophthalmology are typically costly, complex and require trained personnel to operate and are therefore restricted to use in medical facilities such as a doctors office or clinic. This limits the availability of such analysis systems and therefore reduces early detection of eye disorders.

These aspects of conventional approaches monitoring eye disorders make them unsuitable for low cost, convenient home or drugstore use without the intervention of trained personnel. Therefore there is an unmet need for a low cost, convenient and accurate method of detection and monitoring of eye disorders.

### BRIEF SUMMARY OF THE INVENTION

The invention provides a method, apparatus and system for measuring bio-medical attributes of the living eye, such as, internal pressure. The ability to make measurements of the relative location of various surfaces of components of the eye under different conditions is disclosed. The invention includes the ability to apply a pressure disturbance to the eye acoustically and, using non-invasive optical techniques, to perform measurements of vibrations or the time varying relative location of one or more surfaces or structures in a manner correlated with the pressure disturbance. The invention further includes the ability to process the measurements and optionally to compare them with previous measurements to determine such bio-medical attributes as internal pressure, or, for example, the progression of glaucoma or its propensity to occur.

### BRIEF DESCRIPTION OF THE DRAWINGS

FIG. 1 is an illustration of the analysis system according to the invention.

FIG. 2 is a more detailed illustration of aspects of the eye aligned with non-overlapping and overlapping segments of a multiple reference scan according to the invention.

FIG. 3 is a flow chart depicting the steps in an embodiment of the method according to the invention.

### DETAILED DESCRIPTION OF A PREFERRED EMBODIMENT OF THE INVENTION

Conventional analysis systems that detect and monitor eye disorders or the propensity of an eye disorder occurring are typically complex systems that require operation by trained personnel. Furthermore such systems typically each measure only one specific characteristic and therefore multiple systems are typically required.

This invention is a method, apparatus and system for measuring bio-medical attributes of the eye with the ability to make measurements of multiple characteristics of the eye, to do so under different conditions and in a manner such that the measurements can be correlated with the different conditions.

The invention includes the ability to measure the location of multiple surfaces of the eye using a non-invasive optical analysis system based on techniques including, but not limited to, the techniques described in the patent applications incorporated herein by reference.

The invention further includes the ability to measure time varying position of one or more surfaces in response to an applied acoustic or ultrasonic signal. In particular it includes the ability to measure internal pressure of a living eye by applying an acoustic or ultrasonic signal to the eye and measuring the resultant vibrations on the surface of the eye or components of the eye using a optical coherence tomography (OCT) system.

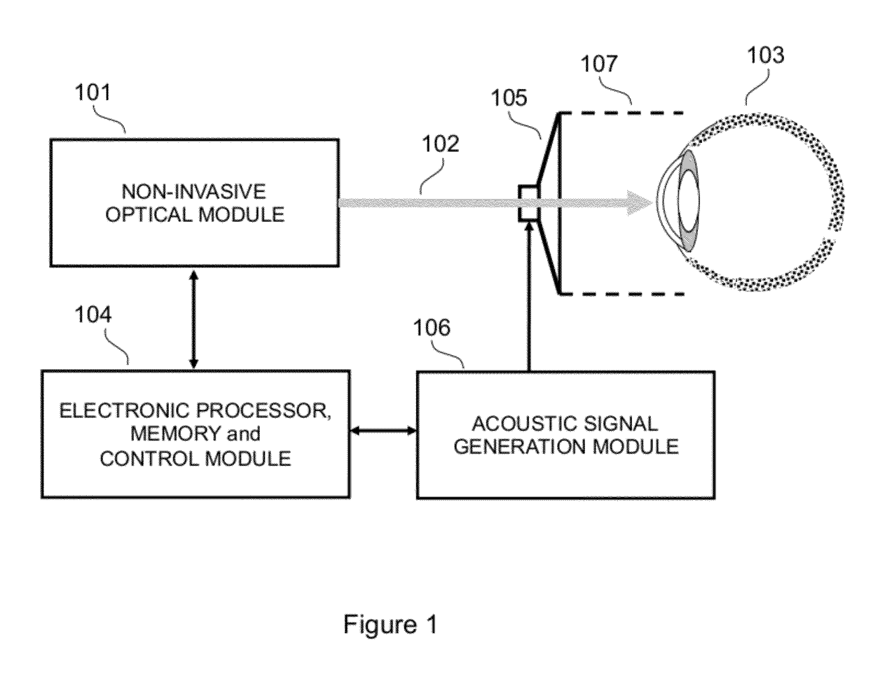

The preferred embodiment is illustrated in and described with respect to FIG. 1. A device for determining internal pressure of a target according to the invention comprises a noninvasive optical module 101, which in the preferred embodiment is an OCT analysis system which measures the time varying relative location of at least one surface of the eye to form time varying relative location information.

The preferred embodiment also includes an acoustic signal generation module 106 and an acoustic or ultrasonic transmitter 105 which together generate a periodic sequence of acoustic waves which are focused onto the target 103. The acoustic waves stimulate vibrations of the target. The frequency and amplitude of vibration are related to structural aspects of the target including internal or intraocular pressure.

A control module 104, which includes a processor, adjusts the frequency content of the acoustic waves. The frequency content of the acoustic waves can be adjusted in any of a number of ways including: adjusting the frequency of a low frequency (ex. hundreds to thousands of Hertz); adjusting the pulse rate of a high frequency acoustic wave (ultrasonic wave) whose frequency can be up to tens of Mega Hertz.

The processor in the control module 104 processes the time varying relative location information to determine the frequency and amplitude content of the time varying relative location information (or vibrations in the target). The characteristics of the vibrations in the target that are related to internal pressure include: frequency and amplitude relationships; values of resonant frequencies; and spatial distribution of modes of vibration. By determining such characteristic of the vibrations on the target in these ways, the internal pressure can be determined.

Further depicted in FIG. 1 is optical probe radiation 102, the target 103 and an optional locating cowl 107 to aid in positioning the device with respect to the eye or other target of interest.

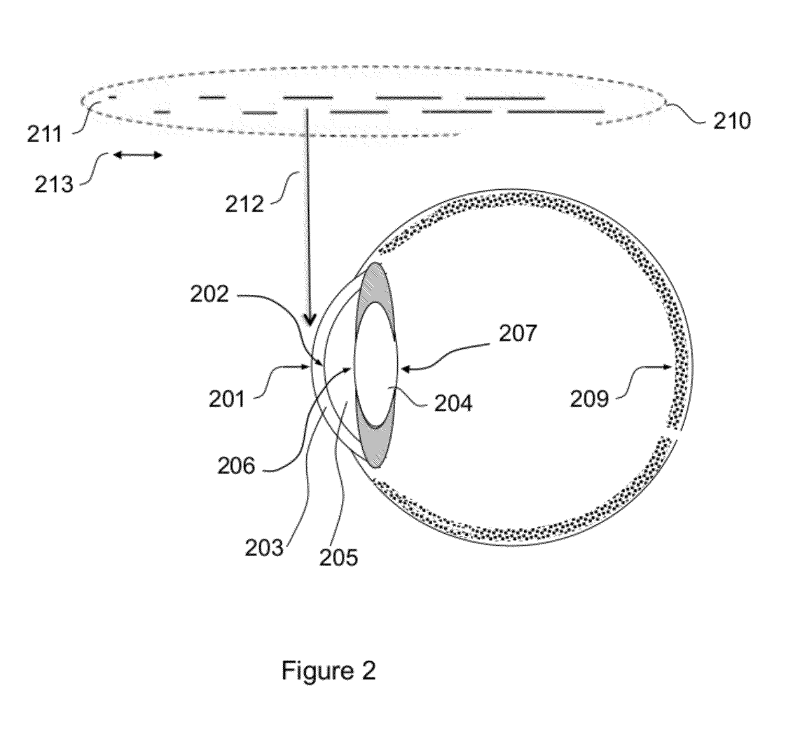

Referring now to FIG. 2 where the target of interest is a living eye (shown as 103 in FIG. 1). When an acoustic or ultrasonic wave (not depicted) is directed at the front surface 201 of the cornea 203 the front surface 201 will vibrate or move in a time varying manner. The nature of the resulting vibrations, or more generally, time varying motion will be related to characteristics of the applied acoustic or ultrasonic wave and the structural characteristics of the eye, including the internal pressure.

In particular, the internal pressure of the eye, at least in part, determines characteristics of the vibrations or time varying motion supported by the eye. Relevant characteristics that are related to the internal eye pressure include, but are not limited to: resonant frequencies and amplitudes; modes of vibration; spatial distribution of vibration amplitudes; nature of decay with time of vibrations.

The vibration or time varying motion of the front surface of the cornea 201 may be measured using optical coherence tomography techniques by measuring its absolute motion or its relative motion with respect to other surfaces within the eye. Suitable surfaces are: the inside surface 202 of the cornea 203; at the inner side of anterior chamber 205, the front surface 206 of the lens 204; the rear surface of the lens 207; and the retinal surface 209. It can be appreciated that any surface naturally occurring or artificially introduced may be useful according to the invention as taught here.

In one embodiment, the absolute motion of the surface 201 may be measured by conventional time domain OCT systems by measuring the Doppler shift of the interference signal frequency. Compensation for relative motion between the analysis system and the eye could be performed by measuring the frequency of the interference signal associated with deeper surfaces, such as 206 or 207 whose Doppler shift (if any) would be associated with relative motion between the analysis system and the eye and not the acoustically stimulated vibration.

In another embodiment, high speed Fourier domain OCT systems (spectral or swept source) could measure the relative motion of the surface 201 by with respect to deeper surfaces, such as 206 or 207, and thereby compensate for relative motion between the analysis system and the eye.

In the preferred embodiment a multiple reference OCT system, described in more detail in the patents and applications incorporated herein by reference, is used as the non-invasive optical module 101 of FIG. 1. As described in the incorporated references the multiple reference OCT system generate optical probe radiation and optical reference radiation and focuses the optical probe radiation within the target, such that at least some of the probe radiation is back-scattered from the target (the eye). The OCT system combines reference radiation with the back-scattered probe radiation, thereby generating interference signals that are related to at least two surfaces of the eye enabling generation of relative motion or location information between the two surfaces.

The acoustic signal generation module 106 applies a compression disturbance to the eye and a timing module (which is included in 104) correlates said relative motion or location information with the acoustic compression disturbance to form correlated time varying relative location information. The acoustic compression disturbance is typically a periodic sequence of acoustic waves that is focused onto the target, thereby stimulating vibration of said target. The frequency content of the acoustic waves is adjusted by, for example, sweeping the frequency of low frequency acoustic waves or sweeping the repetition rate of bursts of ultrasonic waves.

The optical interference signals are detected and processed to determine amplitude and frequency of vibration (or time varying location) in conjunction with timing information related to the swept acoustic signal. With a repetitive swept acoustic signal, phase sensitive techniques can be used to enhance extracting correlated information from the detected interference signals.

In the preferred embodiment at least some of the multiple reference signals of the multiple reference OCT system are aligned with surfaces of the eye under analysis. In one embodiment, illustrated in FIG. 2, a set of multiple reference scan segments are depicted aligned in depth with respect to surfaces of the eye.

The set of ten scan segments, systematically increasing in magnitude, are shown in the dashed oval 210. The first scan segment 211 has a scan magnitude determined by the motion of the scanning piezo device. The references cited herein are commended to the reader desiring supplemental material concerning generation of scans from a multiple reference OCT system. The subsequent scan segments have double, triple, etc, the magnitude of the first scan segment. In this example scan segments from the fifth order and above overlap with adjacent scan segments, thus providing continuous scan information. With respect to FIG. 2, it should be noted that alternate scan segments are depicted offset vertically for illustrative clarity.

As depicted in FIG. 2, the 5^{th} scan segment of the multiple reference signals is aligned with the front surface 201 of the cornea 203 as indicated by the arrow 212. Higher order scan segments 6^{th}, 7^{th}, et cetera, provide continuous scan information relating to the structures at the front of the eye to at least the rear surface 207 of the lens 204.

In the embodiment depicted in FIG. 2, interference signals related to the front surface of the cornea and at least one other surface (such as, for example, the front surface of the lens) can be simultaneously monitored and processed in conjunction with timing information relating to the swept applied acoustic wave to determine amplitude and frequency of vibrations on at least the surface of the cornea. The resulting amplitudes and frequencies are correlated with intraocular pressure.

Processing the interference signals can include, but is not limited to: analyzing Doppler shift information related to different surfaces; compensating for relative motion between the optical analysis system and the eye by extracting Doppler or motion related information common to multiple surfaces; analyzing interference signals from at least two laterally displaced locations to determine the spatial distribution of vibrations; analyzing interference signals from at least two surfaces (displaced in depth) to determine the relative magnitude and phase of vibrations; employing phase sensitive techniques to process the information from the interference signals in conjunction with timing signals related to the swept applied acoustic wave.

In an alternative embodiment the reference radiation associated with the radiation first reflected by a partial reflective mirror in the non-invasive optical module 101 (zero order reference radiation) is aligned with the front surface of the cornea of the eye. The generated baseband interference signal provides information related to the vibration of the front surface of the eye. Higher order interference signals can provide information regarding the location of one or more internal eye surfaces and provide a mechanism for maintaining the zero order reference radiation aligned with the front surface of the eye.

Other structural information, such as the thickness of the cornea or the distance from the front to the retinal (rear) surface of the eye may also be measured and correlated with intraocular pressure. Such measurements may be facilitated by varying the spacing between scan segments of the multiple reference radiation as indicated by 213 of FIG. 2. In such an embodiment one scan segment could be aligned with the front of the cornea while a high order scan is aligned with the retinal surface and at least one intermediate scan segment is aligned with at least one internal eye surface (such as, for example, a surface of the lens).

The processing step may include using known structural aspects of the eye in determining intraocular pressure from vibration information or, alternatively, from time varying location information, where such information is extracted from acquired interference signals. For example, information relating to rigidity of the eye or thicknesses of various components (such as, for example, the cornea) may be included in the processing step using correlation or other techniques.

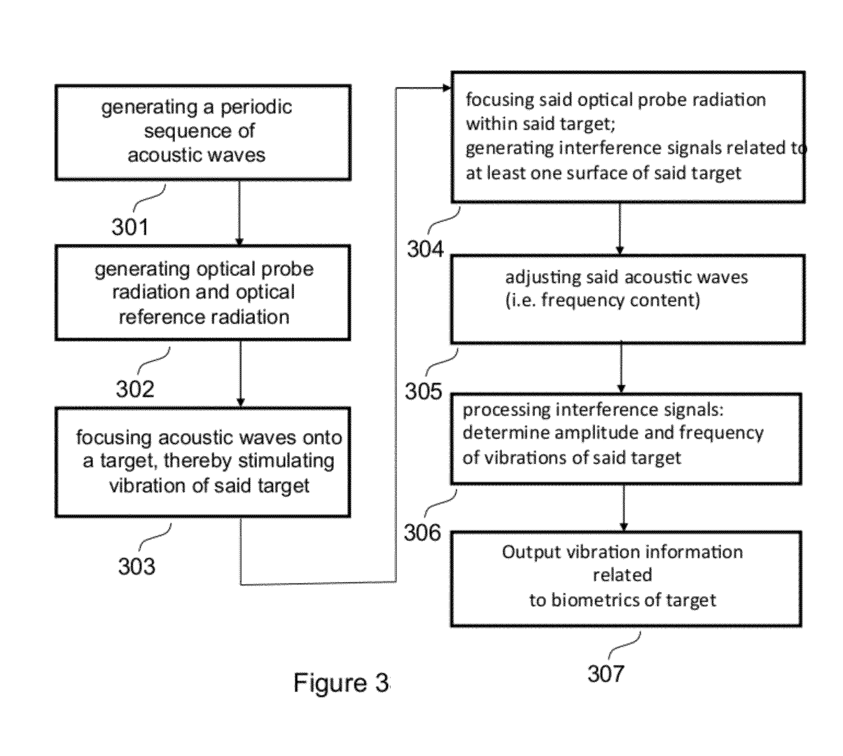

FIG. 3 depicts a method according to the invention. The inventive method comprises the steps of: generating a periodic sequence of acoustic waves (301), generating optical probe radiation and optical reference radiation (302);

focusing the acoustic waves onto the target, thereby stimulating vibration of the target (303); focusing the optical probe radiation within the target, such that at least some of the probe radiation is back-scattered from the target and combining said optical reference radiation with said back-scattered probe radiation, thereby generating interference signals, said interference signals related to at least one surface of said target (304);
adjusting the acoustic waves, wherein the adjusting modifies the frequency content of the acoustic waves (305); and processing the interference signals so as to determine amplitude and frequency of vibrations of the target and where the vibration information is related to the internal pressure, and outputting the vibrational information related to the biometrics of the target (307).

The preferred embodiment the step of generating reference radiation further includes the sub step of generating multiple reference radiation. The step of processing the interference signals further includes the sub step of processing the baseband signal generated by combining backscattered radiation from the front surface of the target with reference radiation first reflected by the partial reflective mirror (i.e. zero order reference radiation), and the baseband signal provides information related to the vibration of the target.

Various embodiments of the inventive method include any of the following steps and substeps: a) where the step of aligning maintains the zero order reference signal aligned with the front surface of said target; b) where the step of processing the interference signals further includes determining relative motion between the target and the optical reference signals; c) where the sub step of processing the baseband signal further includes compensating for relative motion between the target and the optical reference signal; d) where the step of generating the acoustic sequence further includes the sub step of selecting frequency content of the periodic sequences of acoustic waves, including optimizing for target characteristics, when the target is a living eye; e) where the step of aligning further includes the sub step of determining that at least one of the surfaces enables determination of thickness of elements of the target and where the target is a living eye, determining the thickness of the cornea; f) processing the interference signals including compensation of the rigidity of the target.

The above description is intended to be illustrative and not restrictive. Therefore, although many of the features have functional equivalents not set forth comprehensively herein, and variations and combinations not set forth in detail can be readily appreciated by one of average skill in the relevant art, the scope of the invention shall be encompass such functional equivalents, variations and combinations, as such are included in the invention as taught in the specification, claims and accompanying drawings.

For example, in the preferred embodiment a multiple reference OCT analysis system is described. Conventional time domain OCT systems could be used and vibration information extracted using conventional Doppler techniques. Alternatively Fourier domain OCT systems (spectral or swept source) could be used.

It can be appreciated that while for a number of reasons, such as motion compensation, information from at least two surfaces is desirable, it can be appreciated that the invention taught here includes embodiments where information from only one surface is used.

In the preferred embodiment an acoustic wave is generated by a conventional acoustic device. However, a compression disturbance could be generated by a shock wave that could be generated by pulsing optical radiation. Such optical radiation could be the radiation used by the non-invasive analysis system. Such a shock wave could be used instead of or in combination with an acoustic generator.

Other examples will be apparent to persons skilled in the art. The scope of this invention should be determined with reference to the specification, the drawings, the appended claims, along with the full scope of equivalents as applied thereto.

We claim:

1. A method of non-invasively determining internal pressure of a target, said method comprising:
generating a periodic sequence of acoustic waves;
generating optical probe radiation and optical reference radiation;
focusing said acoustic waves onto said target, thereby stimulating vibration of said target;
focusing said optical probe radiation within said target, such that at least some of said probe radiation is back-scattered from said target;
combining said optical reference radiation with said back-scattered probe radiation, thereby generating interference signals, said interference signals related to at least one surface of said target;
adjusting said acoustic waves, wherein said adjusting modifies the frequency content of said acoustic waves; and
processing said interference signals so as to determine amplitude and frequency of vibrations of said target and where said vibration information is related to said internal pressure.

2. The method of claim 1, wherein the step of generating reference radiation further includes the sub step of generating multiple reference radiation;
and where the step of processing said interference signals further includes the sub step of processing the baseband signal generated by combining backscattered radiation from the front surface of the target with reference radiation first reflected by the partial reflective mirror (i.e. zero order reference radiation) such said baseband signal provides information related to the vibration of said target.

3. The method of claim 2 further including the step of aligning some multiple reference signals with at least some surfaces within said target.

4. The method of claim 3 wherein the step of aligning maintains the zero order reference signal aligned with the front surface of said target.

5. The method of claim 1 wherein said step of processing said interference signals further includes determining relative motion between said target and said optical reference signals.

6. The method as in claim 2 wherein the sub step of processing the baseband signal further includes compensating for relative motion between said target and said optical reference signal.

7. The method of claim 1, wherein the step of generating said acoustic sequence further includes the sub step of selecting frequency content of said periodic sequences of acoustic waves, where said sub step of selecting includes optimizing for target characteristics, where said target is a living eye.

8. The method of claim 3 wherein the step of aligning further includes the sub step of determining that at least one of the surfaces enables determination of thickness of elements of said target.

9. The method as in claim 8 wherein said sub step of determining that at least one of the surfaces enables determination of thickness of elements of said target further includes, where said target is a living eye, includes determining the thickness of the cornea of said living eye.

10. The method as in claim 1 wherein said step of processing said interference signals includes compensation for rigidity of said target.

11. The method as in claim 1 wherein said step of focusing said optical probe radiation includes focusing on at least two points, where a first point on the front surface of said target and the second point is some preselected laterally displaced from said first point location on said front surface of said target, and said step of processing includes comparing data from said first point and said second point.

12. The method as in claim 11 wherein said focusing of said probe radiation includes focusing on at least two points, further includes focusing on a first point where said target is a living eye and said first point is on the cornea and where said second point is distal to the center of said cornea, such that the measurement between the first and second point provides vibration information across the eye surface.

13. The method of claim 1 wherein the step of processing said interference signals further includes processing at least two interference signals from at least two surfaces so as to determine amplitude and frequency of vibrations of said target and where said vibration information is related to said internal pressure.

14. A device for determining internal pressure of a target, said device comprising:
an OCT analysis system operable to measure the time varying relative location of at least one surface of said target to form time varying relative location information;
acoustic signal generation module operable to generate a periodic sequence of acoustic waves and to focus said acoustic waves onto said target, thereby stimulating vibration of said target;
a control module, said control module capable of adjusting said acoustic waves, wherein said adjusting modifies the frequency content of said acoustic waves; and
said control module including a processor, said processor capable of processing said time varying relative location information to determine said internal pressure of said target.

15. A device as in claim 14, wherein said OCT analysis system is a multiple reference OCT analysis system.

16. A device as in claim 14, where said device is adapted for a target where said target is living tissue, specifically for a living eye, and further includes:
a component, said component to apply a compression disturbance to said living eye; and
a timing module, said timing module to correlate said relative location information with said compression disturbance to form correlated time varying relative location information.

17. A system for non-invasively determining internal pressure of a living eye, said system comprising:
a component capable of applying a compression disturbance to said eye;
a non-invasive analysis system capable of measuring the relative location of at least two surfaces of said eye so as to generate relative location information of said at least two surfaces;
a timing module, said timing module functioning to correlate said relative location information of said at least two surfaces with said compression disturbance, so as to form correlated relative location information; and
a processing module, said processing module to process correlated relative location information to determine said bio-medical attribute.

18. The system of claim 17, where said non-invasive analysis system is a multiple reference OCT analysis system.
